(19) United States
(12) Patent Application Publication
(10) Pub. No.: US 2013/0190213 A1
(43) Pub. Date: Jul. 25, 2013

(54) Microfluidic Methods, Devices, and Systems for Fluid Handling

(71) Applicant: AFFYMETRIX, INC., Santa Clara, CA (US)

(72) Inventors: Chuan Gao, Sunnyvale, CA (US); Tianyue Yu, San Jose, CA (US)

(73) Assignee: AFFYMETRIX, INC., Santa Clara, CA (US)

(21) Appl. No.: 13/787,413

(22) Filed: Mar. 6, 2013

Related U.S. Application Data
(63) Continuation of application No. 11/553,944, filed on Oct. 27, 2006.
(60) Provisional application No. 60/732,538, filed on Nov. 2, 2005.

(51) Int. Cl. C12Q 1/68

(52) U.S. Cl. CPC C12Q 1/6813
USPC 506/39

(57) ABSTRACT

In one aspect of the invention, systems, methods, and devices are provided for handling liquid. In some embodiments, such systems, methods, and devices are used to process reagent biochemical reactions.

### RELATED APPLICATIONS

The present application claims priority from U.S. Provisional Patent Application Ser. No. 60/732,538, titled “Microfluidic Biological Microarrays”, filed Nov. 2, 2005; which is hereby incorporated by reference herein in its entirety for all purposes.

### BACKGROUND

This application relates to methods, devices, systems for fluid handling. The field of nucleic acid assays has been transformed by microarrays which allow extremely high-throughput and parallel monitoring of gene expression events, expression profiling, diagnostics and large-scale, high-resolution analyses, among other applications. Microarrays are used in biological research, clinical diagnostics, drug discovery, environmental monitoring, forensics and many other fields.

Current genetic research generally relies on a multiplicity of distinct processes to elucidate the nucleic acid sequences, with each process to introducing a potential for error into the overall process. These processes also draw from a large number of distinct disciplines, including chemistry, molecular biology, medicine and others. It would therefore be desirable to integrate the various process used in genetic diagnosis, in a single process, at a minimum cost, and with a maximum ease of operation.

Interest has been growing in the fabrication of microfluidic devices. Typically, advances in the semiconductor manufacturing arts have been translated to the fabrication of micromechanical structures, e.g., micropumps, microvalves and the like, and microfluidic devices including miniature chambers and flow passages.

A number of researchers have attempted to employ these microfabrication techniques in the miniaturization of some of the processes involved in genetic analysis in particular. Conventional approaches often will inevitably involve extremely complicated fluidic networks as more and more reagents are added into systems, for example, greater than 12 reagents for whole transcript assay (WTA) assay, and more samples are processed. By going to a smaller platform, such fluidic complexity brings many concerns such as difficulty in fabrication, higher manufacture cost, lower system reliability, etc. Thus, there's a need to have a simpler way to process samples and perform the reactions in a controlled fashion. However, there remains a need for an apparatus which simplifies and combines the processing of multiple samples and performing the multiple reactions and steps involved in the various operations in the nucleic acid analysis. Various embodiments of the present invention meet one or more of these and other needs.

### SUMMARY OF THE INVENTION

The present invention generally provides methods, devices, systems and computer software products for automated sample preparation of biological assays. The devices, methods and computer software products are suitable for preparing nucleic acid samples for hybridization with microarrays, however, they are not limited to such uses. In one embodiment of the invention, a microfluidic method and system was utilized to produce a sample from a small platform in which the assay introduced several liquids and performed a number of reactions. A device was provided which comprised a housing that contained a liquid cavity. The liquid cavity included a number of inlets that proceeded to a number of inlet channels that were connected to at least one common channel. The common channel was fluidly linked to a number of measuremetric channels which comprise a set of valve mechanisms and gates. The pressure of a gas introduced the liquid into the liquid cavity and by controlling the corresponding valve mechanism(s) and gate(s) the desired volume of liquid was produced. In a preferred embodiment, the housing is made of plastic. In another preferred embodiment, the liquid contains at least one target molecule.

In another preferred embodiment of the present invention, a system as described above was provided wherein the valve mechanism comprised a gas permeable fluid barrier, a valve and housing. The gas permeable fluid barrier comprised a first surface and a second surface, wherein the first surface was exposed to an air cavity. The valve permitted air to flow out of the air cavity. The housing comprised a mounting surface, an air cavity, and a liquid cavity. The liquid cavity comprised an inlet port constructed to permit air flow into the air cavity through said inlet port. The second surface of the gas permeable fluid barrier was sealably mounted with respect to the mounting surface of the housing and the valve was used as the control unit either sealing the cavity or allowing the air to flow. In a preferred embodiment, the gas permeable fluid barrier is a hydrophobic membrane.

Depending upon embodiment, one or more of benefits may be achieved. These benefits and various additional objects, features and advantages of the present invention can be fully appreciated with reference to the detailed description and accompanying drawings that follow.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1a is an image of a system of channels and valves for introducing multiple samples and performing a number of reactions and steps according to an embodiment of the present invention.

FIG. 1b illustrates an example of various volumes.

FIG. 2 illustrates an outline showing the steps to provide a desired volume of liquid into a sample chamber according to an embodiment of the present invention.

FIG. 3 illustrates an alternative embodiment of a valve mechanism design which has an air driven flexible membrane valve.

FIGS. 4a-4d illustrate a method for making and a method for using a valve mechanism with an air driven flexible membrane valve according to an embodiment of the present invention.

FIG. 5 illustrates an alternative embodiment of a valve mechanism design, a 3-layer flexible membrane valve.

FIGS. 6a-6d illustrate a method for making and a method for using a 3-layer flexible membrane valve mechanism according to an embodiment of the present invention.

FIG. 7 illustrates an alternative embodiment of a valve mechanism design, a valve utilizing a gas permeable fluid barrier.

FIGS. 8a-8d illustrate a method for making and a method for using a valve utilizing a gas permeable fluid barrier mechanism according to an embodiment of the present invention.

FIG. 9 illustrates the steps to operate a valve utilizing a gas permeable fluid barrier mechanism according to an embodiment, of the present invention. FIG. 9a illustrates a diagram of the step where the gate is closed and the valve is open according to an embodiment of the present invention. FIG. 9b is a diagram of the step where the gate is opened and the valve is closed according to an embodiment of the present invention.

FIG. 10 illustrates an alternative embodiment of a system of channels and valves for introducing multiple samples and performing a number of reactions and steps.

FIG. 11 illustrates an example of an application with 12 liquids, a WTA Assay.

FIG. 12 illustrates another application, a Microfluidic Card, according to an embodiment of the present invention. FIG. 12a is a layout of a microfluidic card according to an embodiment of the present invention. FIG. 12b illustrates an example of a biochip system.

FIG. 13 illustrates an image of a chip-to-chip interface structure according to an embodiment of the present invention.

### DETAILED DESCRIPTION

The description below is designed to present specific embodiments and not to be construed as limiting in any way. Also, reference will be made to articles and patents to show general features that are incorporated into the present disclosure, but the invention is not limited by these descriptions.

### a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant, techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285 (International Publication Number WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333/179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila, et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592 and U.S. Ser. Nos. 09/916,135, 09/920,491 (U.S. Patent Application Publication 20030096235), Ser. No. 09/910,292 (U.S. Patent Application Publication 20030082543), and Ser. No. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al. Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd ed.,} 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (United States Publication No. 20020183936), Ser. Nos. 10/065,856, 10/065,868, 10/328,818, 10/328,872, 10/423,403, and 60/482,389.

### b) Definitions

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin, beads, silica chips, or other solid supports.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers, initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset, of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion, of a substrate may be subjected to a binary scheme. A combinatorial “masking” strategy is a synthesis which uses light or other spatially selective deprotecting or activating agents to remove protecting groups from materials for addition of other materials such as amino acids.

Effective amount refers to an amount sufficient to induce a desired result.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

Hybridization conditions will typically include salt concentrations of less than about 1M, more usually less than about 500 mM and preferably less than about 200 mM. Hybridization temperatures can be as low as 5 degree C., but are typically greater than 22 degree C., more typically greater than about 30 degree C., and preferably in excess of about 37 degree C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridizations, e.g., allele-specific probe hybridizations, are generally performed under stringent conditions. For example, conditions where the salt concentration is no more than about 1 Molar (M) and a temperature of at least 25 degrees-Celsius (° C.), e.g., 750 mM NaCl, 50 mM NaPhosphate, 5 mM EDTA, pH 7.4 (5×SSPE) and a temperature of from about 25 to about 30° C.

Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2^{nd} Ed. Cold Spring Harbor Press (1989) which is hereby incorporated by reference in its entirety for all purposes above.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics.

Hybridizing specifically to: refers to the binding, duplexing, or hybridizing of a molecule only to a particular nucleotide sequence or sequences under stringent conditions when that sequence is present in a complex mixture (e.g., total cellular) DNA or RNA.

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a Ligand may serve either as the natural Ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

Mixed population or complex population: refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

Monomer: refers to any member of the set of molecules that can be joined together to form an oligomer or polymer. The set of monomers useful in the present invention includes, but is not restricted to, for the example of (polypeptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

mRNA or mRNA transcripts: as used herein, include, but not limited to pre-mRNA transcripts), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic add sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably tyrosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glycosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Probe: A probe is a surface-immobilized molecule that can be recognized by a particular target. See U.S. Pat. No. 6,582,908 for an example of arrays having ail possible combinations of probes with 10, 12, and more bases. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

WGSA (Whole Genome Sampling Assay) Genotyping Technology: A technology that allows the genotyping of hundreds of thousands of SNPS simultaneously in complex DNA without the use of locus-specific primers. In this technique, genomic DNA, for example, is digested with a restriction enzyme of interest and adaptors are ligated to the digested fragments. A single primer corresponding to the adaptor sequence is used to amplify fragments of a desired size, for example, 500-2000 bp. The processed target is then hybridized to nucleic acid arrays comprising SNP-containing fragments/probes. WGSA is disclosed in, for example, U.S. Provisional Application Ser. Nos. 60/319,685; 60/453,930, 60/454,090 and 60/456,206, 60/470,475, U.S. patent application Ser. Nos. 09/766,212, 10/316,517, 10/316,629, 10/463,991, 10/321,741, 10/442,021 and 10/264,945, each of which is hereby incorporated by reference in its entirety for all purposes.

Whole Transcript Assay (WTA): is used herein, a WTA is an assay protocol that can representatively sample entire transcripts (i.e., all exons in a transcript). WTA is disclosed in, for example, U.S. Provisional Application Ser. Nos. 60/683,127 and U.S. patent application Ser. No. 11/419,459, each of which is hereby incorporated by reference in its entirety for all purposes.

Reference will now be made in detail to exemplary embodiments of the invention. While the invention will be described in conjunction with the exemplary embodiments, it will be understood that they are not intended to limit, the invention to these embodiments. On the contrary, the invention is intended to cover alternatives, modification and equivalents, which may be included within the spirit and scope of the invention.

### c) Embodiments

In one aspect of the invention, methods, devices, systems and computer software products for automated sample preparation of biological assays are provided with respect to illustrative, non-limiting, implementations. Various alternatives, modifications and equivalents are possible. The methods, devices, systems and computer software are suitable for performing complex chemical and biochemical reactions. They are particularly suitable for preparing nucleic acids samples for hybridization with microarrays. However, they are not limited to such uses.

For example, certain systems, methods, and computer software products are described herein using exemplary implementations for analyzing data from arrays of biological materials such as, for instance, Affymetrix® GeneChip® probe arrays. However, these systems, methods, and products may be applied with respect to many other types of probe arrays and, more generally, with respect to numerous parallel biological assays produced in accordance with other conventional technologies and/or produced in accordance with techniques that may be developed in the future. For example, the systems, methods, and products described herein may be applied to parallel assays of nucleic acids, PCR products generated from cDNA clones, proteins, antibodies, or many other biological materials. These materials may be disposed on slides (as typically used for spotted arrays), on substrates employed for GeneChip® arrays, or on beads, bead arrays, optical fibers, or other substrates or media, which may include polymeric coatings or other layers on top of slides or other substrates. Moreover, the probes need not be immobilized in or on a substrate, and, if immobilized, need not be disposed in regular patterns or arrays. For convenience, the term “probe array” will generally be used broadly hereafter to refer to all of these types of arrays and parallel biological assays.

The device of the present invention is generally capable of carrying out a number of preparative and analytical reactions on a number of samples. In a preferred embodiment, to achieve this end, the device generally comprises a number of inlet channels, a common channel and a set of control valves within a single unit or body.

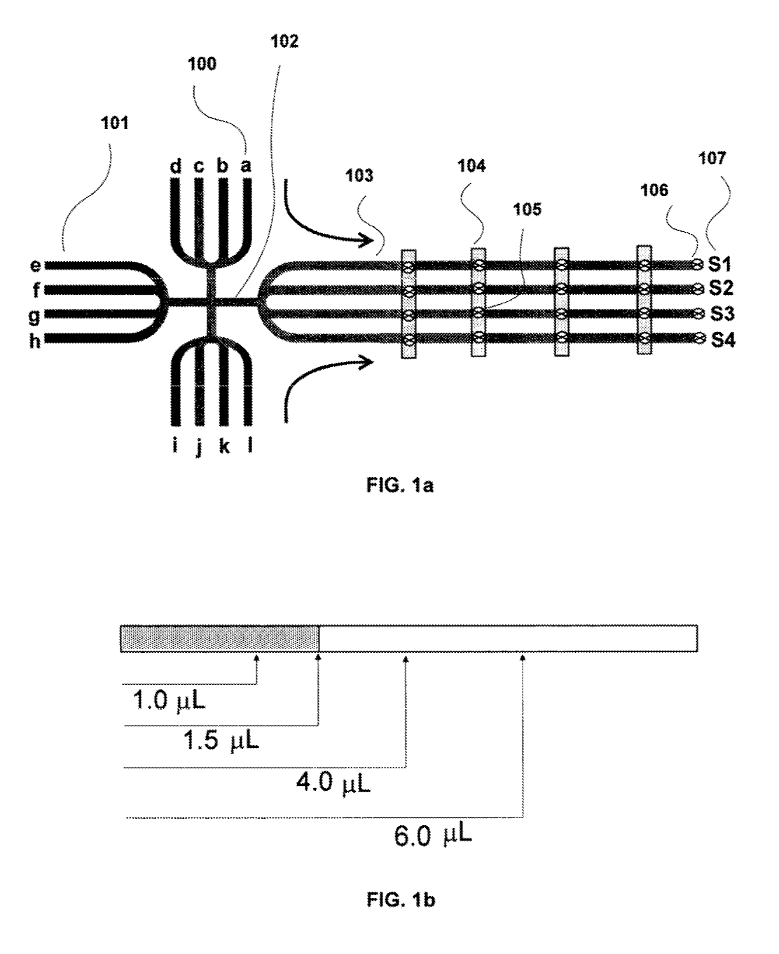

According to one aspect of the present invention, a system for introducing multiple samples and performing multiple reactions and steps is provided as shown in FIG. 1a. This diagram is merely an example, which should not unduly limit the scope of the claims. One of ordinary skill in the art would recognize many variations, alternatives, and modifications. This adjustable microfluidic splitting structure is used to deliver various volumes of liquids into a number of sample chambers. This system includes a housing that comprises a liquid cavity that is made up of a plurality of inlet channels that are fluidly connected at a common channel. A liquid is introduced into the inlet of an inlet channel. In a preferred embodiment, the valves are controllable such that the valves are activated to divide the liquid into a plurality of measuremetric channels and provide a desired volume of liquid. This system as mentioned earlier can be utilized in various applications. Specific volumes of multiple of liquids may be processed to provide a multiple number of samples. In one preferred embodiment, the liquid contains at least one target molecule.

Typically, the body of the device defines the various inlet channels, common channel(s) and measuremetric channels in which the above described operations are carried out according to certain embodiments of the present invention. Fabrication of the body and thus the various channels and chambers disposed within the body may generally be carried out using one or a combination of a variety of well known manufacturing techniques and materials as described in U.S. Pat. Nos. 6,197,595 and 6,830,936. These references are incorporated herein by reference in its entirety. The body of the device is generally fabricated using one or more of a variety of methods and materials suitable for micro fabrication techniques such as embossing, injection molding, thermal bonding thermal forming, etc. Typical plastic materials used for microfluidics are thermal-plastics: polycarbonate, poly methyl methacrylate (PMMA), COC, etc. and elastomers: polydimethylsiloxane (PDMS). For example, in a preferred embodiment, the body of the device may be injected molded parts from Polycarbonate.

As shown in FIG. 1a, liquids (100 a to l) are loaded into the system from the inlets of the inlet channels (101), which are the channels that are used to transfer the liquid from the inlet to the common channel (102). In general, the dimensions of the channels within the miniaturized device may be embodied in any number of shapes depending upon the particular need. Additionally, these dimensions will typically vary depending upon the number of liquids, the number of reactions performed and the number of samples and the like. Typically, the number of fluidic channels is equal to the number of reagents multiplied by the number of samples. In one aspect of the present invention, the number of fluidic channels is equal to the sum of the number of reagents and the number of samples. In a preferred embodiment, after the liquids are introduced, the liquids pass through one common channel (102). The liquid may split up into the various measuremetric channels (103). As discussed above, the channels may be of various dimensions, shapes and quantities. There may be a set (104) of individual valves (105) that control the fluid flow of the liquids into the specific channels. For each measuremetric channel, different valve locations may correspond to different volumes. For the valve, different channel may correspond to the same volume or different volumes. In another preferred embodiment, where there are different volumes, controllable valves are provided. Computer software products are provided to control various active components (i.e. the valves, or liquids, microfluidic system, etc.), temperature and measurement devices. The system can be conveniently controlled by any programmable device, preferably a digital computer such as a Dell personal computer. The computers typically have one or more central processing unit coupled with a memory. A display device such as a monitor is attached for displaying data and programming. A printer may also be attached. A computer readable medium such as a hard drive or a CD ROM can be attached. Program instructions for controlling the liquid handling can be stored on these devices.

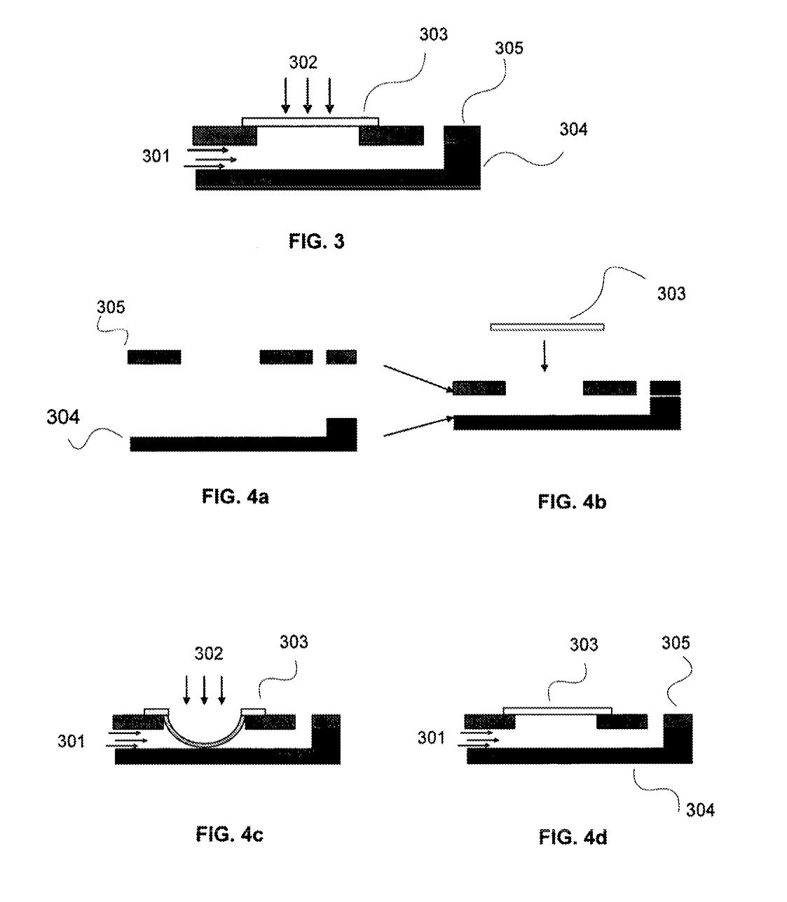

In another preferred embodiment, a measuremetric channel and a valve mechanism may be used to precisely measure fluid volumes for introduction into a subsequent sample chamber. In such cases the location of the valve mechanism of the channel will be dictated by measuremetric needs of a given reaction. Further, the measuremetric channel(s) may be fabricated to include a number of valve mechanisms to provide a number of volumes. In a preferred embodiment, the controllable valves will stop the liquid at a location to provide the desired volume. FIGS. 3-9 illustrate preferred embodiments of three valve mechanism designs. Combination of different valve locations can realize variant volume dispensing. FIG. 1b provides an example of various volumes that could be specified by the location of the valves. This diagram is merely an example, which should not unduly limit the scope of the claims. One of ordinary skill in the art would recognize many variations, alternatives, and modifications. In this example, valves 1-4 correspond to volumes of 1.0 μl, 1.5 μl, 4.0 μl and 6.0 μl respectively. As mentioned above, the number of valves will be dependent on several factors, for example, the size of the platform, the number of different volume requirements, etc. In general, the measuremetric channels will be from about 0.05 μl to about 20 μl in volume, preferably from about 1.0 μl to 10 μl. The desired volume of liquid may be provided by activating the valves (105) and gates (106) of the channels. There can be a number of sets (104) of valves depending on the volume requirements of the liquids to produce the samples (107) in the corresponding sample chambers.

According to an embodiment of the present invention, an apparatus for providing a plurality of predetermined volumes of a liquid includes a first plurality of channels, each of the first plurality of channels capable of holding a volume of a liquid, and a second plurality of channels directly or indirectly connected to the first plurality of channels. The second plurality of channels is coupled to a plurality of valves, and each of the second plurality of channels includes a plurality of channel segments. A first segment of the plurality of channel segments is connected to a second segment of the plurality of channel segments if at least one of the plurality of valves is closed, and the first segment of the plurality of channel segments is disconnected from the second segment of the plurality of channel segments if the at least one of the plurality of valves is open. For example, the apparatus is implemented according to FIG. 1a and/or FIG. b.

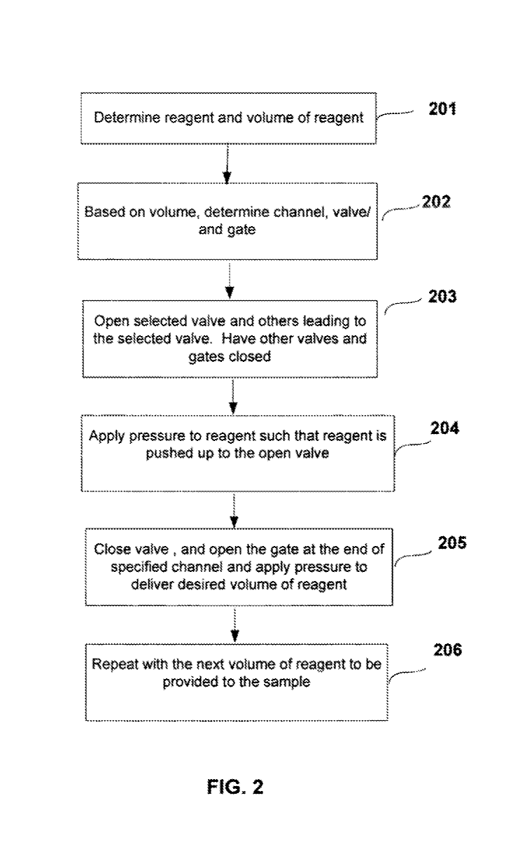

In one aspect of the invention, FIG. 2 illustrates an outline showing the steps of one preferred method to provide a desired volume of a liquid (100) into a sample chamber (107) that provides a sample (107). This diagram is merely an example, which should not unduly limit the scope of the claims. One of ordinary skill in the art would recognize many variations, alternatives, and modifications. In FIG. 1, a-l represent liquids and S1, S2, S3, and S4 represent four different samples. The first step (process 201) is to determine the volume of liquid (100) needed for the reaction. At the process 202, based on the volume, the channels, valves and gates that, will be used in the operation will be determined. At the process 203, the selected valve and others leading to the selected valve will be opened while keeping the other valves and gates closed. The next step (process 204) is to apply pressure to the liquid such that liquid is pushed up to the open valve. At the process 205, once the liquid reaches the desired destination, the valve is closed and the gate at the end of the specified channel is opened. Air or gas pressure is further applied to deliver the desired volume of liquid. At the process 206, the steps (e.g., processes 201-205) are repeated to deliver the next volume of liquid to the sample.

In a preferred embodiment, a liquid is usually provided to all the samples in the same or various quantities. The smallest volume required of any of the liquids may be indicated by the first valve. When the pressure is applied, the pressure can be applied equally such that volume of liquid is equally split between the channels. This process may be accomplished based on the design of the valve mechanism, the operation of the microfluidics and the characteristics of the liquid.

Another embodiment of the present invention is the air-driven microfluidics method. Filtered pressured air is used as the driving force and is regulated by high precision pressure regulators. In general, the high precision pressure regulators range will be from about 0 psi to about 5 psi, preferably from about 0 psi to about 2 psi. Computer controlled mini air valves may be used to control the air flow and integrated pressure sensors for pressure recording. The movement of liquid is controlled by air or gas pressure and valve open time. Examples of microfluidic valves as well as fluid flow and control is discussed in, for example, Paul C. H. Li, Microfluidic Lab-on-a-chip for Chemical and Biological Analysis and Discovery, 2006, and A. van den (Albert) Ber, et al, Lab-on-Chips for Cellomics, 2004 and Oliver Geschke, et al, Microsystem Engineering of Lab-on-a-chip Devices, 2004, each of which is hereby incorporated by reference in its entirety for all purposes.

According to yet another embodiment, a method for providing a plurality of predetermined volumes of a liquid includes providing a volume of a liquid to a channel. The channel is directly or indirectly connected to a plurality of channels, and the plurality of channels is coupled to a plurality of valves. Each of the plurality of channels includes a plurality of channel segments, and a first segment of the plurality of channel segments is capable of being connected to or disconnected from a second segment of the plurality of channel segments in response to at least one of the plurality of valves. Additionally, the method includes receiving information associated with a plurality of predetermined volumes for the liquid corresponding to the plurality of channels respectively, and processing information associated with the plurality of predetermined volumes for the liquid. Moreover, the method includes selecting one valve from the plurality of valves based on at least information associated with the plurality of predetermined volumes, opening the selected valve, and transporting the liquid through the plurality of channels up to the opened valve. For example, the method is implemented according to FIG. 2. In another example, the method also includes closing the selected valve after the transporting the liquid through the plurality of channels up to the opened valve. The process for closing the selected valve is performed so that the liquid flows in the plurality of channels, and the plurality of channels holds the plurality of predetermined volumes of the liquid respectively. In yet another example, the process for transporting the liquid through the plurality of channels up to the opened valve includes applying a pressure to the liquid in the channel.

FIGS. 3, 5 and 7 illustrate alternative embodiments of valve mechanism designs: a valve design with an air driven flexible membrane, a 3-layer flexible membrane valve design, and a valve design utilizing a gas permeable fluid barrier respectively. These diagrams are merely examples, which should not unduly limit the scope of the claims. One of ordinary skill in the art would recognize many variations, alternatives, and modifications.

FIG. 3 illustrates an alternative embodiment of a valve mechanism design which has an air driven flexible membrane valve. As shown in FIG. 3, the air driven flexible membrane valve design is simple such that it is made up of a channel formed by plastic (304 and 305) and a flexible membrane. The flexible membrane may be composed of any material that will be able to function as described in this application. In a preferred embodiment, the flexible membrane is a polydimethylsiloxane (PDMS) membrane (303).

FIGS. 4a-4d illustrate a method for making and a method for using a valve mechanism with an air driven flexible membrane valve according to an embodiment of the present invention. These diagrams are merely examples, which should not unduly limit the scope of the claims. One of ordinary skill in the art would recognize many variations, alternatives, and modifications. As shown in FIG. 4a, this design includes two layers of plastic (304 and 305) which are bonded together as shown in FIG. 4b. The flexible membrane (303) is then bonded to the second layer of plastic (305). In a preferred embodiment, the bonding is performed with an adhesive. The second layer of plastic (305) is preferably made out of a plastic that is compatible with the flexible membrane. Air pressure (301) is used to push the liquid through the channels, while the valve mechanism is used to control the volume of liquids by stopping the liquid at a desired location. As shown in FIG. 4c, the air or gas pressure (302) is used to activate the valve by pressing against the flexible membrane (303). When the air pressure (302) pressing against the flexible membrane is greater than the air pressure (301) pushing the liquid, the flexible membrane (303) is pushed such that it protrudes into the channel, blocking the gate. To clear the gate, the air pressure (302) is turned off as shown in FIG. 4d.

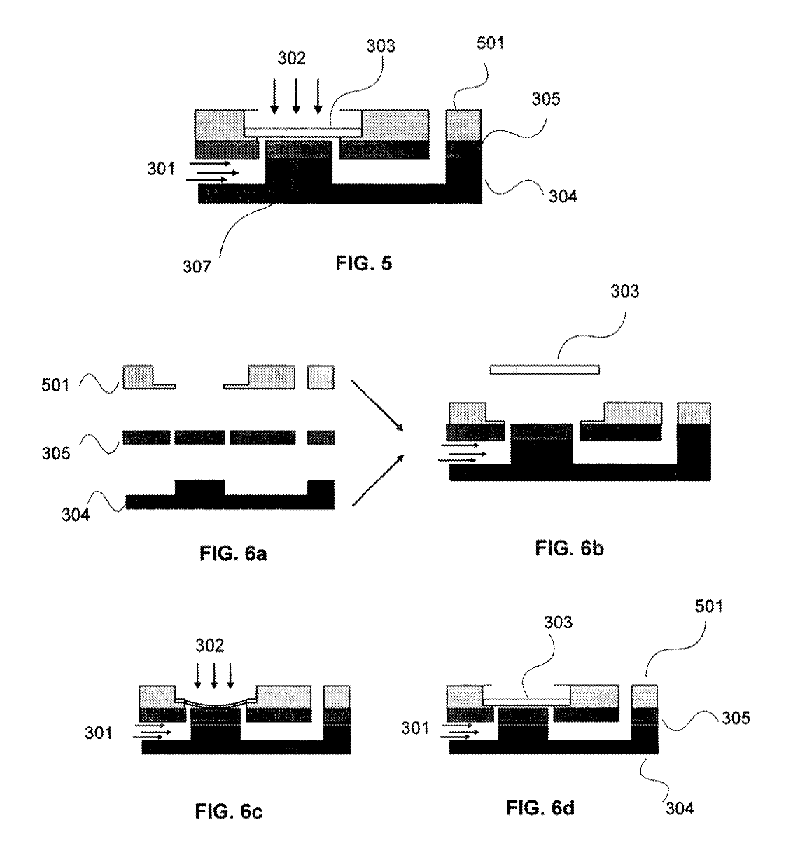

FIG. 5 illustrates an alternative embodiment of a valve mechanism design, a 3-layer flexible membrane valve mechanism. FIGS. 6a-6d illustrate a method for making and a method for using a 3-layer flexible membrane valve mechanism according to an embodiment of the present invention. These diagrams are merely examples, which should not unduly limit the scope of the claims. One of ordinary skill in the art would recognize many variations, alternatives, and modifications.

As shown in FIG. 6a, this design is composed of 3 layers: a plastic layer (304) to form the channels, a second layer of plastic (305) to be able to form a liquid tight seal against the flexible membrane (303), and a third layer of plastic (501) to support the flexible membrane (303) in place. The three plastic layers are bonded together as shown in FIG. 6b. In a preferred embodiment, as discussed above, the bonding is performed with an adhesive. A flexible membrane (303) as mentioned above is bonded to the third plastic layer as shown in FIG. 6b. This design introduces a protrusion feature (307) in the first plastic layer (304). A second layer of plastic is bonded to the first layer to form the protrusion feature (307). While the air pressure (301) is pushing the liquid through the channel, the air pressure (302) pushes the flexible membrane against the protrusion feature (307) as shown in FIG. 6c to stop the flow of liquid. Thus, the second layer is made out of a material that is compatible with the flexible membrane (303). The protrusion feature (307) may be of any shape, material such that when pressure is applied it stops the liquid from flowing. To clear the gate, the air pressure (302) is turned off as shown in FIG. 6d.

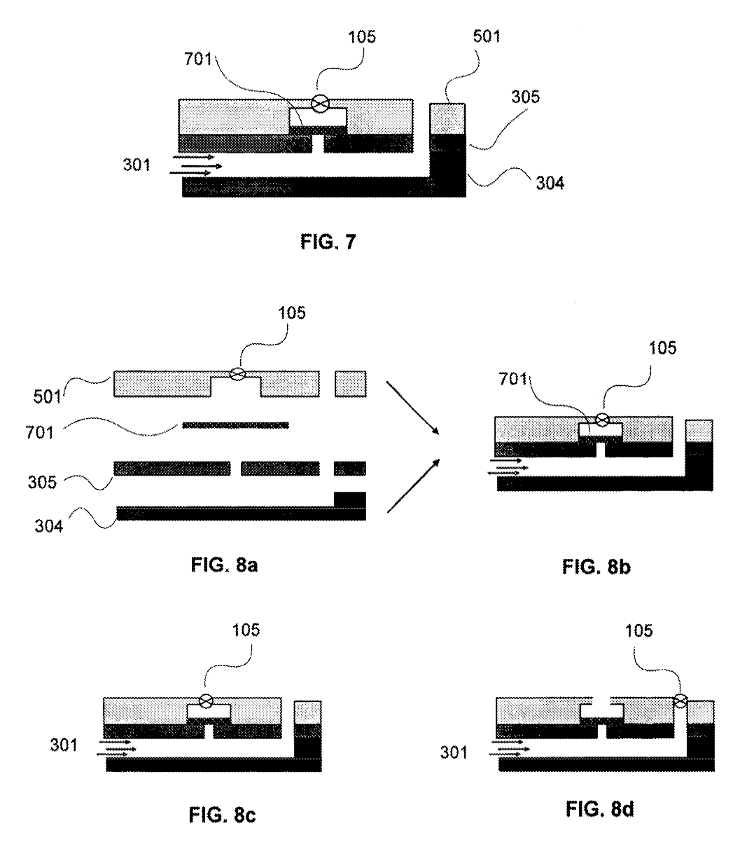

FIG. 7 illustrates another alternative embodiment of a valve mechanism design, a valve utilizing a gas permeable fluid barrier. In a preferred embodiment, this design includes a gas permeable fluid barrier (308) and a valve (105). The gas permeable fluid barrier is a barrier which permits the passage of gas without allowing for the passage of fluid. A variety of materials are suitable for use as a gas permeable fluid barrier including, e.g., porous hydrophobic polymer materials, such as spun fibers of acrylic, polycarbonate, Teflon, pressed polypropylene fibers, or any number commercially available gas permeable fluid barrier (GE Osmonics labstore, Millipore, American Filtrona Corp., Gelman Sciences, and the like).

In a preferred embodiment, FIGS. 8a-8d illustrate a method for making and a method for using a valve utilizing a gas permeable fluid barrier mechanism. These diagrams are merely examples, which should not unduly limit the scope of the claims. One of ordinary skill in the art would recognize many variations, alternatives, and modifications. As shown in FIG. 8a, this design is also composed of 3 layers: a plastic layer (304) to form the channels, a second layer of plastic (305) to be able to form a liquid tight seal against the gas permeable fluid barrier (701), and a third layer of plastic (501) to support the gas permeable fluid barrier (701) in place. The three plastic layers and the gas permeable fluid barrier (701) are assembled and bonded together as shown in FIG. 8b. In a preferred embodiment, as discussed above, the bonding is performed with an adhesive. A gas permeable fluid barrier (701) as mentioned above is bonded to the second plastic layer as shown in FIG. 8b. The air pressure (301) may push the liquid through the channel while the valve (105) is closed and the gate (106) is open as shown in FIG. 8c. The movement of the liquid is stopped when the valve (105) is closed and the gate (106) is opened as shown in FIG. 8d. In a preferred embodiment, the measuremetric channels and valve design is such that the gas permeable fluid barrier is not contacted with the liquid.

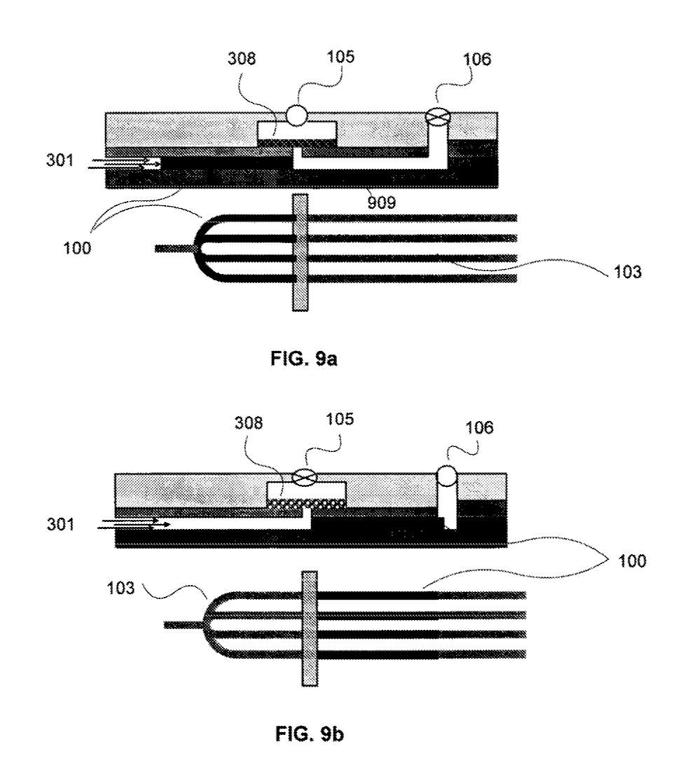

An illustration of another embodiment of how the method with the valve utilizing a gas permeable fluid barrier operates is shown in FIG. 9a and 9b. These diagrams are merely examples, which should not unduly limit the scope of the claims. One of ordinary skill in the art would recognize many variations, alternatives, and modifications. There can be several ways one can operate a system that is presented. In a preferred embodiment, multiple liquids can be added sequentially to various numbers of samples simultaneously. In one aspect of the present invention, an example of how a liquid can be added to various numbers of samples simultaneously is shown in FIGS. 9a and 9b.

As shown in FIG. 9a, while the gates (106) are closed and the valves (105) are opened, the air pressure (301) fills all the measuremetric channels (103) and pushes the liquid through the measuremetric channels (103). The introduction of the liquid displaces the gas present in the channel. The gas permeates through the gas permeable fluid barrier until the liquid (100) reaches the desired location (909). The liquid is held in place by utilizing surface tension. Then the gates (106) are opened and the valves (105) are closed as shown in FIG. 9b. The air pressure (301) pushes the liquid (100) through the measuremetric channels (103). In a preferred embodiment, the liquid can be prevented from being added to a sample by closing the corresponding gate (106) and valve (105) which will prevent the liquid from filling that specific measuremetric channel.

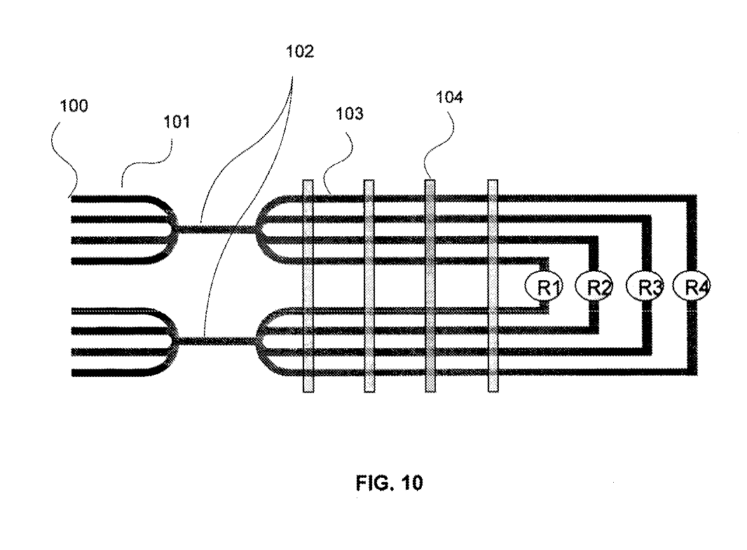

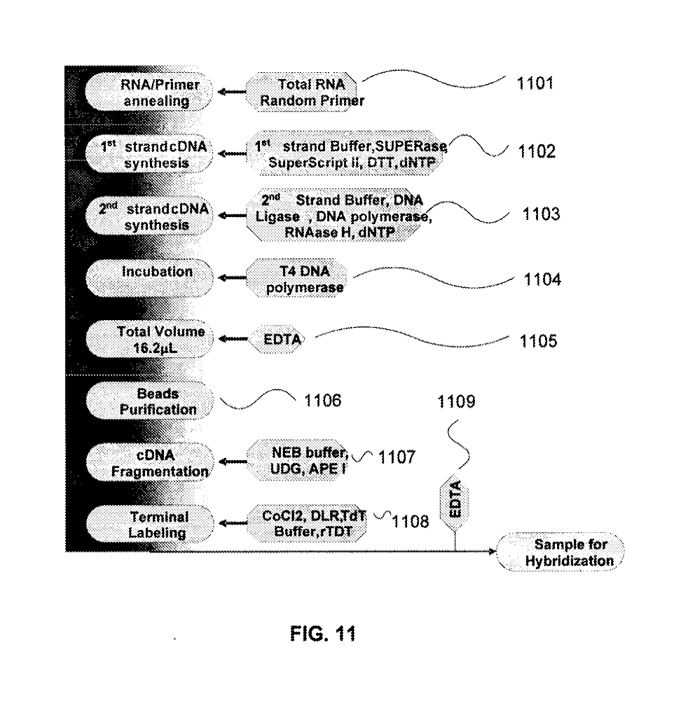

In another preferred embodiment, a schematic of another system for introducing multiple samples and performing a number of reactions and steps is shown in FIG. 10. This diagram is merely an example, which should not unduly limit the scope of the claims. One of ordinary skill in the art would recognize many variations, alternatives, and modifications. In this system, there can be a number of liquids (100) and corresponding inlet channels (101). All the inlet channels (101) can be connected by a number of common channels (102). In this example there are two common channels (102) which separate into two sets of measuremetric channels that lead to corresponding sample chambers that perform a number of separate reactions. In this example, there are four reactions: R1, R2, R3, and R4 and four sets of valves (104). As discussed previously, the number of samples, reactions, sets of valves, etc. will depend on several factors such as the application.

In one aspect of the invention, a system is provided for splitting a plurality of liquids comprises a housing which comprises a liquid cavity. The liquid cavity comprises a plurality of inlet channels, at least one common channel, a plurality of measuremetric channels, a computer and a controlling device. The inlet channels comprise a plurality of inlets which are fluidly connected by at least one common channel. The common channel is fluidly linked to a number of measuremetric channels which comprise a set of valve mechanisms and gates. The pressure of a gas introduces the liquid into a measuremetric channel. A computer is used with the controlling device to control the valve mechanisms and gates such that the liquid is split into the plurality of measuremetric channels and the desired volume of liquid is produced. In a preferred embodiment, the housing is made of plastic. In another preferred embodiment, the liquid contains at least one target molecule.

In another preferred embodiment of the present invention, a system as described above is provided wherein the valve mechanism comprises a gas permeable fluid barrier, a valve and housing. The gas permeable fluid barrier comprises a first surface and a second surface, wherein the first surface is exposed to an air cavity. The valve permits air to flow out of the air cavity. The housing comprises a mounting surface, an air cavity, and a liquid cavity. The liquid cavity comprises an inlet port constructed to permit air flow into the air cavity through said inlet port. The second surface of the gas permeable fluid barrier is sealably mounted with respect to the mounting surface of the housing. The valve was used as the control unit either sealing the cavity or allowing the air to flow. In a preferred embodiment, the gas permeable fluid barrier is a hydrophobic membrane.

In one aspect of the present invention, an apparatus for controlling liquids is provided which comprises a gas permeable fluid barrier and a valve. The gas permeable fluid barrier comprises a first surface and a second surface, such that the first surface is exposed to an air cavity. The valve permits air to flow out of the air cavity. The housing comprises a mounting surface, the air cavity, and a liquid cavity. The liquid cavity comprises an inlet port constructed to permit air flow into said air cavity through said inlet port. The second surface of the gas permeable fluid barrier is sealably mounted with respect to the mounting surface of the housing whereby the valve is located inside the air cavity. In a preferred embodiment, the apparatus as described above is provided wherein the housing is made of plastic. In another preferred embodiment, the apparatus as described above is provided wherein the gas permeable fluid barrier is a hydrophobic membrane.

In another aspect of the present invention, a method for controlling liquids is provided which comprises providing a gas permeable fluid barrier which comprises a first surface and a second surface, the first surface exposed to an air cavity. The method then involves providing a valve, wherein the valve permits air to flow out of the air cavity, sealably mounting the second surface of the gas permeable fluid barrier to a housing which comprises a mounting surface, the air cavity, and a liquid cavity. The liquid cavity comprises an inlet port constructed to permit air flow into said air cavity through said inlet port, wherein the sealably mounting step assist in preventing a liquid to pass through the gas permeable fluid barrier. The method continues by controlling the valves to introduce the liquid inside the liquid cavity and stopping the liquid at a desired location. In a preferred embodiment, the method described above is provided wherein the housing is made of plastic. In another preferred embodiment, the method described is provided wherein the gas permeable fluid barrier is a hydrophobic membrane.

A system for controlling liquids is provided which comprises a plurality of devices where there is a first device and a second device. The first device comprises an outlet port and the second device comprises an inlet port. A gasket comprises a first surface and a second surface, wherein the first surface is flat around the inlet port to assure a liquid tight seal when mated to the second device. The housing comprises an alignment and a clamping mechanism to align and clamp the first device and second device together. The compression of the gasket permits liquid to flow from the first device to the second device. In a preferred embodiment, the system describe above is provided wherein the housing is made of plastic. In another preferred embodiment, the system described above is provided wherein the first device comprises a heating device. In another preferred embodiment, the system described above is provided wherein the first device comprises a mixing device. In another preferred embodiment, the system described above is provided wherein the second device comprises a probe array.

Most assays require multiple reagents to be added to multiple reactions. In many embodiments, the liquid is a reagent for a biochemical reaction. FIG. 11 illustrates an example of an application with 12 reagents, a WTA Assay. This diagram is merely an example, which should not unduly limit the scope of the claims. One of ordinary skill in the art would recognize many variations, alternatives, and modifications.

As shown in FIG. 11, this WTA assay provides several reactions that need various numbers of reagents. The assay begins with the first sample. Total RNA (1101). The next step (1102) adds the second reagent, 1st strand buffer where the 1^{st} strand cDNA is synthesized. The 2^{nd} strand cDNA is then synthesized when the third reagent is added. During incubation (1103), the fourth reagent is added. A total volume of 16.2 μl (1105) is achieved when the fifth reagent, EDTA. The beads purification step involves 4 reagents: magnetic beads, alcohol, alcohol, and water (1106). The cDNA fragmentation (1107) is completed when the 10^{th} reagent is added. After, the eleventh reagent is added to performed the Terminal Labeling step. The last reagent, EDTA, is added to provide a sample for hybridization. A total of 12 reagents are involved in this WTA assay. This sample may then be hybridized with an Affymetrix U133A chip.

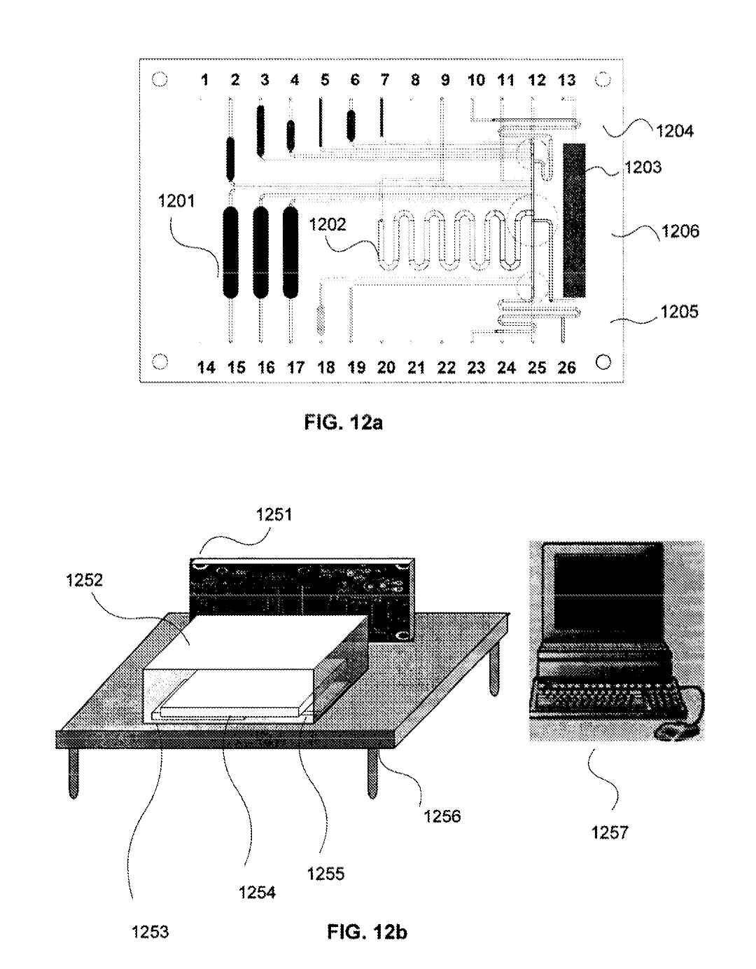

FIG. 12a illustrates another application which is an example of a layout of a microfluidic card, according to an embodiment of the present invention. This diagram is merely an example, which should not unduly limit the scope of the claims. One of ordinary skill in the art would recognize many variations, alternatives, and modifications. The ports are identified as numbers 1-26 in FIG. 12a. Port 6 performs the primer annealing mix, port 4 performs the 1^{st} strand synthesis, and port 3 performs the 2^{nd} strand synthesis. Port 5 performs the T4 DNA polymerase, port 7 adds the EDTA, and port 2 adds the water. Port 15 introduces the beads solution, and port 16 and 17 provides the alcohols. Port 18 performs the fragmentation, port 19 performs the labeling, and port 20 adds the EDTA. Ports 11, 12, and 25 are the chamber vents. Ports 9, 10, and 23 are used for mixing. Port 13 is the waste port, and port 26 is the collection port. The microfluidic card design layout includes storage chambers (1201), mixing channels (1202) and a waste chamber (1203). In addition to the chambers and channels, this design also includes heating devices (1204 & 1206) and a magnet (1206).

In one aspect of the invention, a cartridge device is provided. The reagents can be stored in a measuremetric microfluidics system with controllable valves, wherein the valves will stop the fluids at desired locations. Computer software products are provided to control the various active components (i.e., fluidic structure, valves, etc.). This system to operate a microfluidic card which may also be called a biochip system is shown in FIG. 12b according to an embodiment of the present invention. This diagram is merely an example, which should not unduly limit the scope of the claims. One of ordinary skill in the art would recognize many variations, alternatives, and modifications. A circuit board (1251) is provided for functions, for example, temperature control. The manifold (1252) includes a mechanical valve, pressure sensor, pressure regulator, etc. biochip or microfluidic card (1254) is installed into the manifold (1252) where there is a cooling unit (1253) and a heating/magnetic unit (1255). Both the circuit board (1251) and the manifold (1252) with all its components are mounted on a base plate (12560). A computer (1257) is used for several functions, for example, controlling the valves.

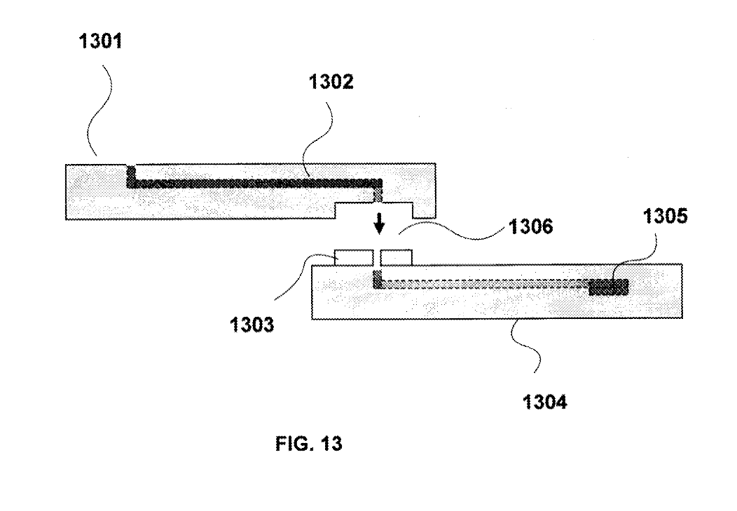

In another aspect of the present invention, the biochip system may comprise of a number of devices or cards. There may be several different types of cards. FIG. 13 illustrates an image of a chip-to-chip interface structure according to an embodiment of the present invention. This diagram is merely an example, which should not unduly limit the scope of the claims. One of ordinary skill in the art would recognize many variations, alternatives, and modifications.

For example, a reagent cards (1301) may include a plurality of inlets, a plurality of various channels, valve mechanisms and an outlet as discussed previously in this application. Examples of a reaction card are sample prep, target prep and the like. In another embodiment, reaction cards (1304) that receive the sample(s) from reagent card(s) are provided. Examples of a reaction card are hybridization, wash, stain, scanning, reading and the like. In a preferred embodiment, a reaction card comprises a probe array (1305) or a chamber that can receive a probe array (1305). In a preferred embodiment, the biochip to chip or card to card interface structure is a gasket (1303) to perfect fitting. The gasket (1303) may be mounted on the reaction card.

In another embodiment, an example of how a biochip system operates is provided. A reaction card with the gasket may be installed into the manifold (1252) as indicated in FIG. 12b. The reagent card is mated over the reaction card. In a preferred embodiment, alignment pins are provided to assure that the cards are properly aligned. The manifold may have a clamping mechanism to press the two cards together.

The device, systems and methods of the present invention has a wide variety of uses in the manipulation, identification and/or sequencing of nucleic acid samples according to certain embodiments of the present invention. These samples may be derived from plant, animal, viral or bacterial sources. For example, the device, method and system of the invention may be used in diagnostic applications, such as in diagnosing genetic disorders, as well as diagnosing the presence of infectious agents, e.g., bacterial or viral infections. Additionally, the device, method and system be used in a variety of characterization applications, such as forensic analysis, e.g., genetic fingerprinting, bacterial, plant or viral identification or characterization, e.g. epidemiological or taxonomic analysis, and the like.

The present invention provides devices, methods and systems for liquid handling according to certain embodiments of the present invention. It is to be understood that the above description is intended to be illustrative and not restrictive. Many variations of the invention will be apparent to those of skill in the art upon reviewing the above description and figures. All cited references, including patent and non-patent literature, are incorporated herein by reference in their entirety for all purposes.

The invention will be further understood by the following non-limiting examples.

### EXAMPLES

### Example 1

### Steps to Produce 1.5 μl of Reagent (a) to Sample 1

In an experiment, 1.5 μl of reagent (a) was provided to the first sample, S3. The following are steps that were performed to accomplish this task. As shown in FIG. 1a, the third channel leads to S3, therefore, the valves and gates connected to the third channel were controlled to provide 1.5 μl of reagent (a) into S3. The 2^{nd} valve on the third channel was identified to provide 1.5 μl as shown in FIG. 1b and shown as (105) in FIG. 1a. The valve (105) and the others leading to valve (105) were opened while making sure that the other valves and gates were closed. Pressure was applied to reagent (a) such that reagent (a) was pushed up to the open valve (105). Next, the valve (105) was closed and the gate at the end of third channel was opened. Air pressure was applied and the desired volume of reagent (a) was provided to S3.

### Example 2

### System with Multiple Reagents and Multiple Reactions

Experiments were performed to optimize the WTA assay protocol such that the introduction of the number of reagents and the multiple reactions can be performed on a miniature platform device. FIG. 11 illustrates an example of the results, showing the WTA assay protocol layout with 12 reagents to produce a sample for hybridization. In this example, as shown in FIG. 11, this WTA assay protocol provided several reactions that needed various numbers of reagents. The assay began with 1 sample, Total RNA (1101). Next, the second reagent, 1st strand buffer (1102) was added and the 1^{st} strand cDNA was synthesized. After the 2^{nd} strand cDNA was synthesized, with the addition of the third reagent, the solution was incubated (1103) with the addition of the fourth reagent. EDTA, the fifth reagent, was added to make a total volume of 16.2 μl (1105). The beads purification step involved four reagents: magnetic beads, alcohol, alcohol, and water (1106). Afterwards, cDNA fragmentation (1107) was completed with the addition of the tenth reagent. The eleventh reagent was added and the Terminal Labeling step was completed. Finally, the last reagent, EDTA, was added and the resulting sample was then hybridized with an Affymetrix U133A chip. A total of 12 reagents were used in this WTA assay protocol.

### Example 3

### Device with Multiple Reagents and Multiple Reactions

A device as described in the present application was created based on the assay described in Example 2. An illustration of an example of a layout of a microfluidic card is shown in FIG. 12a. The microfluidic card was designed, with 26 ports, indicated as numbers 1-26 in FIG. 12a. In performing the experiments, Port 6 performed the primer annealing mix, port 4 performed the 1^{st} strand synthesis, and port 3 performed the 2^{nd} strand synthesis. Port 5 performed the T4 DNA polymerase, port 7 added the EDTA, and port 2 added the water. Port 15 introduced the beads solution, and port 16 and 17 provided the alcohols. Port 18 performed the fragmentation, port 19 performed the labeling, and port 20 added the EDTA. Ports 11, 12, and 25 were the chamber vents. Ports 9, 10, and 23 were used for mixing. Port 13 was the waste port, and port 26 was the collection port. The microfluidic card design layout included storage chambers (1201), mixing channels (1202) and a waste chamber (1203). In addition to the chambers and channels, this design also included heating devices (1204 & 1206) and a magnet (1206).

The overall system to operate a microfluidic card or a biochip system is shown in FIG. 12b. A circuit board (1251) was provided for functions, for example, temperature control. The manifold (1252) included a mechanical valve, pressure sensor, pressure regulator, etc. biochip or microfluidic card (1254) was installed into the manifold (1252) where there was a cooling unit (1253) and a heating/magnetic unit (1255). Both the circuit board (1251) and the manifold (1252) with all its components were mounted on a base plate (12560). A computer (1257) was used for several functions, for example, controlling the valves.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

1.-20. (canceled)

21. A microfluidic device for performing biological assays, the microfluidic device comprising:
a plurality of inlet channels configured to introduce a plurality of liquids into the microfluidic device;
at least one common channel connected to the plurality of inlet channels;
at least one chamber; and
a plurality of measuremetric channels connected to the at least one common channel, wherein the plurality of measuremetric channels are configured to measure and deliver a volume of at least one of the plurality of liquids into the at least one chamber.

22. The microfluidic device of claim 21, wherein the plurality of measuremetric channels include a plurality of valve mechanisms.

23. The microfluidic device of claim 22, wherein the plurality of valve mechanisms are configured to control liquid flow through the plurality of measuremetric channels.

24. The microfluidic device of claim 23, wherein at least one of the plurality of valve mechanisms comprises an air driven flexible membrane.

25. The microfluidic device of claim 24, wherein the air driven flexible membrane comprises a channel and a flexible membrane.

26. The micro fluidic device of claim 23, wherein at least one of the plurality of valve mechanisms comprises a three-layer flexible membrane.

27. The microfluidic device of claim 26, wherein the three-layer flexible membrane comprises a first layer configured to form a channel, a second layer configured to form a seal against a flexible membrane, and a third layer configured to bond to the flexible membrane.

28. The microfluidic device of claim 23, wherein at least one of the plurality of valve mechanisms comprises a gas permeable fluid barrier.

29. The microfluidic device of claim 28, wherein the gas permeable fluid barrier comprises a barrier that permits passage of gas without permitting passage of fluid.

30. The microfluidic device of claim 21, wherein the plurality of measuremetric channels are configured to measure and deliver from 0.05 microliters to 20 microliters of volume.

31. The microfluidic device of claim 30, wherein the plurality of measuremetric channels are configured to measure and deliver from 1 microliter to 10 microliters of volume.

32. The microfluidic device of claim 31, wherein the plurality of measuremetric channels are configured to measure and deliver 1 microliter, 1.5 microliter, 4 microliter, and 6 microliter quantities.

33. The microfluidic device of claim 21, wherein the microfluidic device comprises a plastic material.

34. The microfluidic device of claim 33, wherein the plastic material is selected from the group consisting of polycarbonate, polymethyl methacrylate, and polydimethylsiloxane.
